(19) United States
(12) Patent Application Publication
(10) Pub. No.: US 2008/0003652 A1
(43) Pub. Date: Jan. 3, 2008

(54) Novel enzymatic process for the manufacture of Boc-Dap-Oh priority to related application(s)

(76) Inventors: Hans Iding, Rheinfelden (DE); Rudolf Schmid, Rheinfelden (CH); Rene Trussardi, Birsfelden (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/818,768

(22) Filed: Jun. 14, 2007

(30) Foreign Application Priority Data
Jun. 28, 2006 (EP) 06116203.8

(51) Int. Cl. C12P 17/10

(52) U.S. Cl. 435/121

(57) ABSTRACT

The present invention relates to a process for making a compound of formula (I)

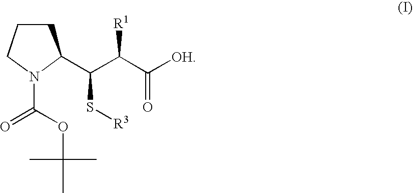

The process involves the use of an enzyme. Compounds of formula (I) are intermediates in the manufacture of Dolastatin 10 analogues, which are useful in the treatment of cancer.

### PRIORITY TO RELATED APPLICATION(S)

This application claims the benefit of European Patent Application No. 06116203.8, filed Jun. 28, 2006, which is hereby incorporated by reference in its entirety.

### BACKGROUND OF THE INVENTION

The present invention relates to a new, enzymatic process for the manufacture of derivatives of 3-pyrrolidin-2-yl-propionic acid.

The compounds obtainable by the process according to the present invention are valuable intermediates in the manufacture of Dolastatin 10 analogues. Dolastatin 10 is known to be a potent antimitotic peptide, isolated from the marine mollusk Dolabella auricularia, which inhibits tubulin polymerization and is a different chemical class from taxanes and vincas (Curr. Pharm. Des. 1999, 5: 139-162). Preclinical studies of Dolastatin 10 have demonstrated activities against a variety of murine and human tumors in cell cultures and animal models. Dolastatin 10 and two synthetic dolastatin derivatives, Cemadotin and TZT-1027, are described in Drugs of the future 1999, 24(4): 404-409.

Subsequently it had been found that certain Dolastatin 10 derivatives having various thio-groups at the dolaproine part show significantly improved anti-tumor activity and therapeutic index in human cancer xenograft models (WO 03/008378). However the synthesis disclosed in WO 03/008378 suffers from low yields, mainly due to laborious separation of the diastereoisomer mixtures, obtained in the β-addition reaction, by chromatography. Therefore there remains a need to provide new and improved processes for the synthesis of Dolastatin 10 derivatives, including new and improved processes for the synthesis of intermediates used in the process of making such derivatives.

The present invention addresses this problem by providing a new, improved process for making a compound of the general formula (I) (described subsequently), which is a key intermediate in the synthesis of the above-mentioned Dolastatin 10 derivatives. More precisely, it has now surprisingly been found that the enzymatic process of the present invention provides an improved diastereoisomer ratio and an improved yield of the compound of formula (I). Furthermore the process according to the present invention avoids the laborious separation of the diastereoisomer mixtures by chromatography.

### SUMMARY OF THE INVENTION

One aspect of the present invention is a process for making a compound of formula (I)

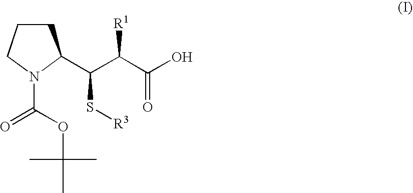

which comprises

### (A) reacting a compound of formula (II)

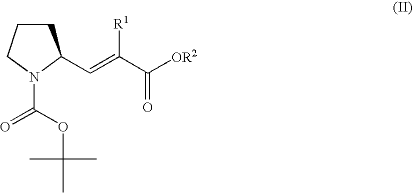

with a compound of formula (III)

KS—R³ (III),

in the presence of triethylammonium chloride in a suitable solvent, to obtain a diastereomeric mixture of formula (IV)

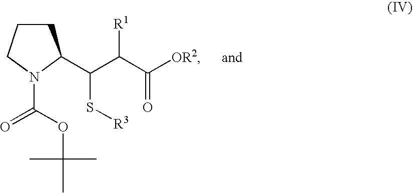

### (B) cleaving, using a hydrolase, R² from the —COOR² ester group of the diastereoisomer of the above diastereomeric mixture that has the formula of formula (IVa)

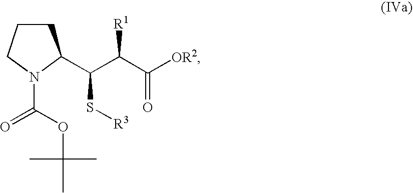

to obtain a compound of formula (I).

### R¹ in the above formulas is methyl or ethyl which can be substituted, once or several times, by fluorine; or unsubstituted propyl.

### R² in the above formulas is alkyl.

### R³ in the above formulas is methyl or ethyl.

The compound of formula (III) may be generated in situ by reacting a compound of formula (III-A),

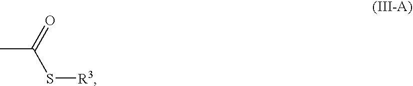

in which R³ is methyl or ethyl, in the presence of a potassium base.

Another aspect of the present invention is a process for making a compound of formula (A),

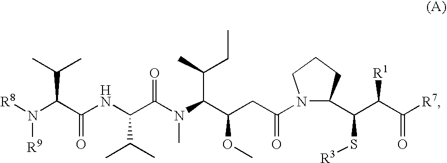

which comprises:
(1) reacting a compound of formula (I), made using the above process, with a compound of the formula R⁷H;
(2) cleaving the tert-butoxycarbonyl group contained in the resulting compound at the pyrrolidine N-atom, to produce a compound of formula (B),

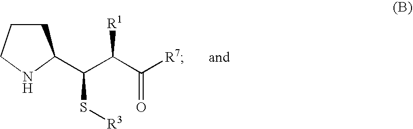

(3) reacting a compound of formula (B) with a compound of formula (C),

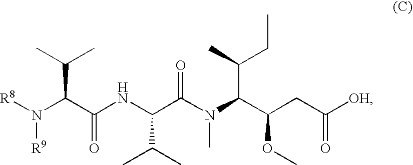

to produce the compound of formula (A).

### R¹ in the above formulas is methyl or ethyl which can be substituted, once or several times, by fluorine; or unsubstituted propyl.

### R² in the above formulas is alkyl.

### R³ in the above formulas is methyl or ethyl.

### R⁸ and R⁹ in the above formulas are each independently hydrogen or C₁₋₄ alkyl.

### R⁷ in the above formulas is phenylalkylamine, phenyldialkylamine, or phenylalkyloxy; wherein the alkyl group in phenylalkylamine, phenyldialkylamine, or phenylalkyloxy is a C₁₋₄ alkyl; and

the phenyl group in phenylalkylamine, phenyldialkylamine, or phenylalkyloxy may optionally be substituted with one, two or three substituents selected from the group consisting of halogen, alkoxycarbonyl, sulfamoyl, alkylcarbonyloxy, carbamoyloxy, cyano, mono- or di-alkylamino, alkyl, alkoxy, phenyl, phenoxy, trifluoromethyl, trifluoromethoxy, alkylthio, hydroxy, alkylcarbonylamino, 1,3-dioxolyl, 1,4-dioxolyl, amino and benzyl.

The above, and other objects, features, and advantages of the present invention will become apparent from the following description read in conjunction with the accompanying drawings.

### BRIEF DESCRIPTION OF THE DRAWINGS

FIG. 1 depicts the amino acid sequence for ESP-ESL-1083 [SEQ ID NO: 1].

FIG. 2 depicts the nucleic acid sequence [SEQ ID NO: 2] which encodes ESP-ESL-1083.

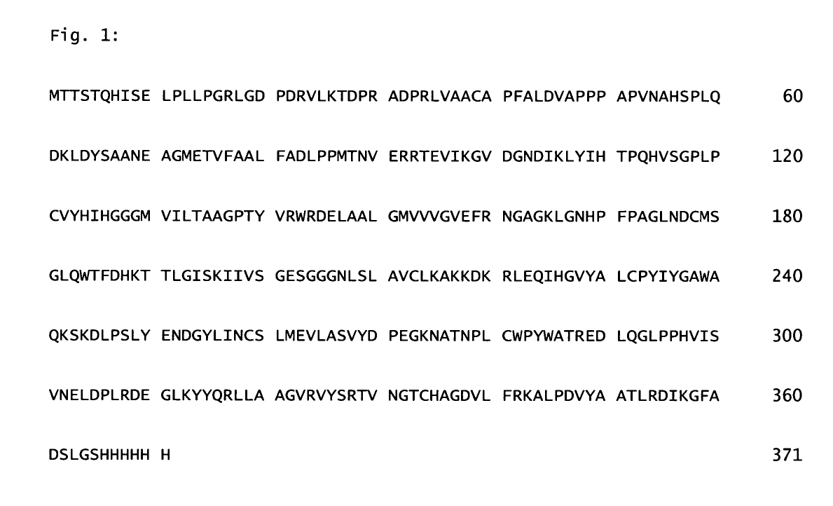

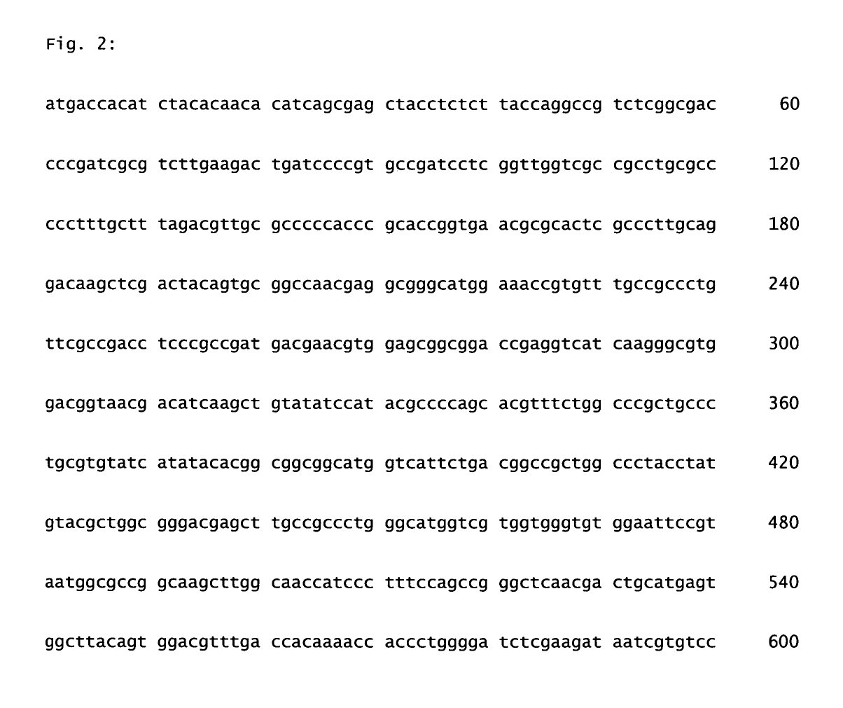

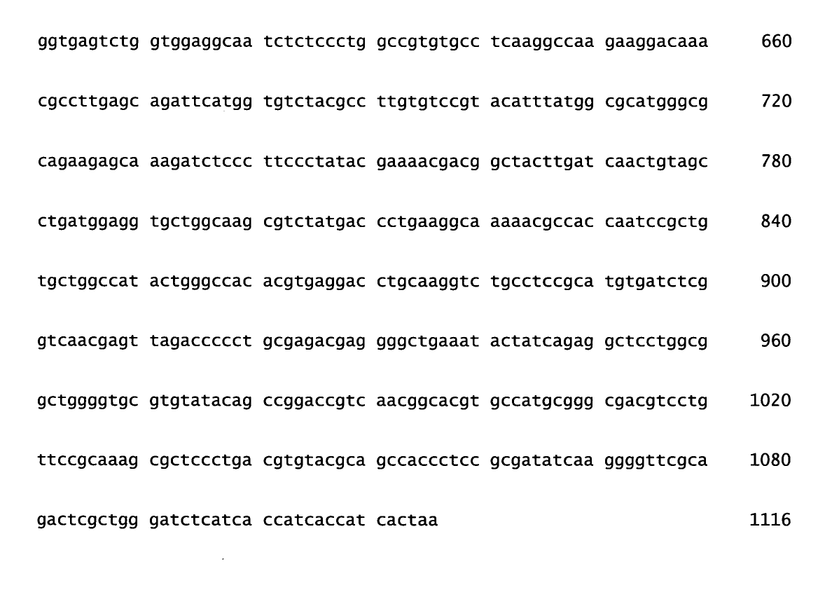

### DESCRIPTION OF THE SEQUENCE LISTING

SEQ ID NO: 1 is the amino acid sequence for ESP-ESL-1083.

SEQ ID NO: 2 is a nucleic acid sequence which encodes ESP-ESL-1083 [SEQ ID NO: 1].

### DETAILED DESCRIPTION OF THE INVENTION

As used herein, the term “alkyl”, when used alone or used in describing a group comprising an alkyl (for example, phenylalkyl, alkylthio) refers to a straight-chain or branched-chain hydrocarbon group containing a maximum of 8, preferably a maximum of 6, carbon atoms, e.g., methyl, ethyl, n-propyl, n-butyl, 3-methylbutyl, n-pentyl, 3-methylpentyl, 4-methylpentyl, or n-hexyl; more preferably a maximum of 4 carbon atoms. A “C₁₋₄ alkyl” group is an alkyl group, as defined above, containing a maximum of 4 carbon-atoms. An alkyl group may be unsubstituted or may be substituted with one or more substituents, preferably with one to three substituents, most preferably with one substituent. The substituents are selected from the group consisting of hydroxyl or halogen.

As used herein, the term “alkoxy”, when used alone or used in describing a group comprising an alkoxy (for example, alkoxycarbonyl), refers to a substituent of the formula alkyl-O— with “alkyl” being as defined above.

The term “halogen” refers to fluorine, bromine, iodine and chlorine, preferably fluorine and chlorine.

The term “potassium base” refers to a potassium-containing compound with a pH-value above 7 in aqueous media, such as potassium hydroxide or potassium alkoxides, especially potassium ethoxide.

The term “hydrolase” refers to enzymes that catalyze hydrolysis reactions.

The term “esterase” refers to a hydrolase that catalyzes the hydrolysis of esters.

The term “stereoselective”, when used in reference to a hydrolase, refers to a hydrolase (including an esterase) that acts predominantly on a specific stereoisomer (e.g., a diastereoisomer).

The present invention relates to a process for making a compound of formula (I) (hereafter, also referred to as “Process I”).

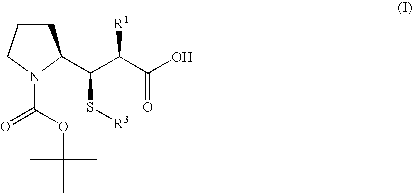

Process I comprises the steps of:

### (A) reacting a compound of formula (II)

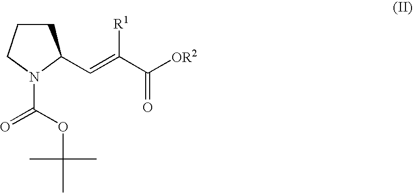

with a compound of formula (III)

KS—R³ (III),

in the presence of triethylammonium chloride in a suitable solvent, to obtain a diastereomeric mixture of formula (IV)

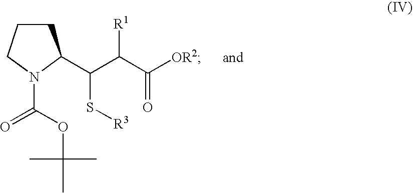

### (B) cleaving, using a hydrolase, R² from the —COOR² ester group of the diastereoisomer of the above diastereomeric mixture that has the formula of formula (IVa)

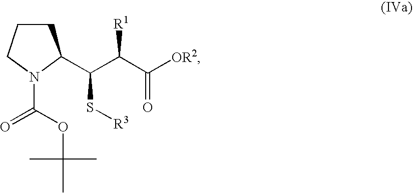

to obtain a compound of formula (I).
R¹ in the above formulas is methyl or ethyl which can be substituted, once or several times by fluorine; or unsubstituted propyl. If the methyl or ethyl group of R¹ is substituted, it is preferably mono- or di-substituted, more preferably mono-substituted.

### R² in the above formulas is alkyl.

### R³ in the above formulas is methyl or ethyl.

In an embodiment of the present invention, the compound of formula (III) is generated in situ by reacting a compound of formula (III-A),

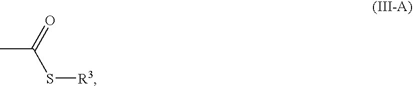

in which R³ is methyl or ethyl, in the presence of a potassium base.

Another embodiment of the present invention is a process of Process I in which R¹ and R³ are both methyl.

A further embodiment of the present invention is a process of Process I in which R¹ and R³ are both methyl and R² is ethyl.

Yet another embodiment of the present invention is a process of Process I in which R¹ and R³ are both methyl; R² is methyl, ethyl, propyl, or butyl; and the hydrolase is an amino acid of SEQ ID NO: 1.

The hydrolase used in the process of the present invention is an enzyme which is capable of cleaving the R² from the —COOR² ester group of the compound of formula (IVa). In an embodiment of the present invention, the hydrolase is an esterase. In a preferred embodiment, the hydrolase is a stereoselective hydrolase which acts predominantly on the diastereoisomer of the formula (IVa). In a more preferred embodiment, the stereoselective hydrolase is an esterase (a stereoselective esterase). Examples of preferred stereoselective esterases include ESP-ESL-1083, also known as BD 1083, which is the amino acid sequence of SEQ ID NO: 1, and variants thereof. ESP-ESL-1083 may be purchased from the company Diversa Corporation having a registered address at 4955 Directors Place, San Diego, Calif. 92121, U.S.A. General methods for obtaining and isolating such enzymes are inter alia described in WO 02/057411. It has now surprisingly been found that, out of enzymes screened, solely the esterase having the amino acid sequence of SEQ ID NO: 1 displays a high stereoselectivity. SEQ ID NO: 2 is a nucleic acid sequence that encodes the amino acid sequence of SEQ ID NO: 1. Accordingly, another preferred embodiment of the present invention involves the use of a protein that is encoded by SEQ ID NO: 2 or a variant of such a protein.

The term “variant” in this context relates to a protein that is: (A) a product formed by the degradation of the protein having the amino acid sequence of SEQ ID NO: 1 (hereafter “protein of SEQ ID NO: 1), for example a product of proteolytic degradation of the protein of SEQ ID NO: 1 which may still be recognized by diagnostic means or by ligands directed against the protein of SEQ ID NO: 1; (B) splice variants of the protein of SEQ ID NO: 1; and (C) a protein that has an amino acid sequence that is substantially similar to that of SEQ ID NO: 1. As used in the present application, the term “substantially similar” refers to an amino acid sequence that is at least 80% similar to the amino acid sequence of SEQ ID NO: 1. In preferred embodiments, the variant has an amino acid sequence that is at least 85%, more preferably at least 90%, and most preferably at least 95%, similar to the amino acid sequence of SEQ ID NO: 1.

A “suitable solvent”, for use in the practice of the present invention, will depend upon the reaction steps used in the present process. The aforementioned step (A) is preferably carried out in an ether, for example, tetrahydrofuran, methyl-tetrahydrofuran, tert-butyl methyl ether, dimethylether, or diethylether, and at temperatures from −20° C. to the reflux temperature of the respective solvent, most preferably from 0° C. to room temperature. The aforementioned step (B) may be carried out with suitable enzymes in aqueous reaction media. In this connection aqueous media also means suspensions and/or emulsions of poorly water soluble compounds in water. As a common alternative, the hydrolase used in step (B) may also be used in an immobilized form. Such “immobilized forms” are well known alternatives to the person of ordinary skill in the art.

The following general reaction scheme (scheme 1) describes a process which involves the steps of the aforementioned Process I. Unless explicitly otherwise stated, R¹, R² and R³ have the significances given herein before. “Boc”, as used herein, refers to tert-butoxycarbonyl, “Ph” refers to phenyl, and “Et” refers to ethyl.

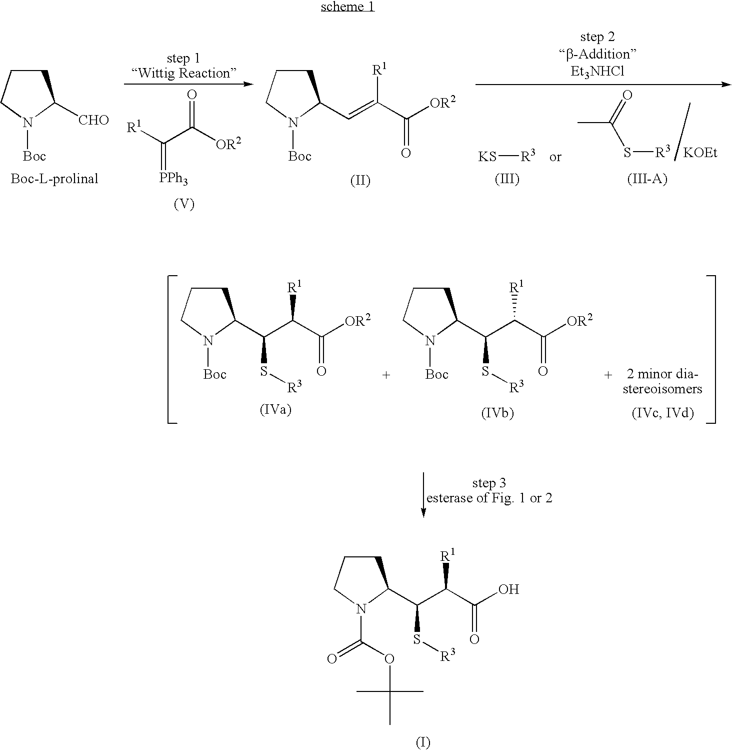

Step 1: This step demonstrates a step for making a compound of formula (II), which is a starting compound for the process of the aforementioned Process I. Step 1 represents a Wittig reaction in which commercially available tert-butoxycarbonyl protected L-prolinal (Boc-L-prolinal) is reacted with a ylide (V) and using methods known to the skilled artisan (see e.g. Heterocycles, 36 (9), 1993, 2073-2080 and WO 03/008378) to form a compound of formula (II).

Step 2: This reaction is a β-addition of alkyl-mercaptanes, especially methyl mercaptane, wherein the potassium salt of formula (III) can be used as such, or generated in situ by adding the compounds of formula (III-A) in the presence of potassium bases, for example potassium ethoxide. The use of triethylammonium chloride (Et₃N×HCl) as the proton source is especially preferred. The reaction leads to a mixture of diastereoisomers, preferably one in which the diastereoisomer of formula (IVa) is predominantly present.

Step 3: This reaction is a diastereomerically selective ester cleavage. The treatment of an emulsion of the diastereoisomer mixture of the compounds of formula (IV) (compounds of formulas IVa, IVb, IVc, IVd) with the enzyme having the amino acid sequence of SEQ ID NO: 1 leads to a highly stereoselective ester cleavage of diastereoisomer of formula (IVa) to produce a compound of formula (I). The substrate is applied in concentration of 1-5%, preferably around 2-3%. A suitable reaction temperature is room temperature to 35° C., a suitable reaction pH is between 6.5 and 8.5. As to the aqueous part of the emulsion, common buffer solutions known to be used for biochemical conversions are used, for example, phosphate or Tris-buffer in a concentration of 3-300 mM, preferably 3-100 mM. Such a buffer can additionally contain a salt like e.g. NaCl and Na₂SO₄ in a concentration of 50 mM to 1M or LiSCN in a concentration of 50 mM to 500 mM, a polyhydric alcohol like glucose in a weight percentage of 2-20%, polyethylene ether in a weight percentage of 2-25% or a water miscible organic solvent like ethanol in a volumetric percentage of 2-10%. The additives may increase the solubility of the compound of formula (IV) or increase the stability of the esterase. After the addition of the enzyme, the pH of the reaction mixture is maintained while stirring by the controlled addition of base such as NaOH or KOH, whereby the formed acid goes into solution and the reaction mixture becomes rather clear. After termination of the reaction, unreacted diastereomeric esters are extracted from the reaction mixture and subsequently the aqueous phase is acidified. The acid formed thereby (a compound of formula (I)) is extracted with a common organic solvent. The compound of formula (I) can then be obtained and/or purified by crystallization from organic solvents, preferably from hexane or heptane.

Still another embodiment of the present invention is a process for making a compound of formula (A).

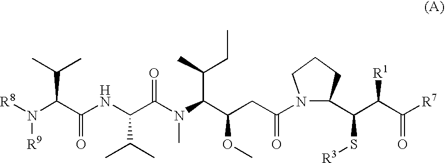

The process comprises the step of the above-described Process I and further comprises the steps of:
(a) reacting the thus produced compound of formula (I) with a compound of the formula R⁷H;
(b) cleaving the tert-butoxycarbonyl group contained in the resulting compound at the pyrrolidine N-atom, to produce a compound of formula (B),

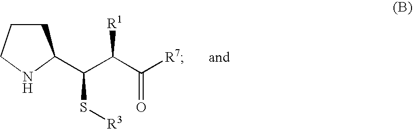

(c) reacting a compound of formula (B) with a compound of formula (C),

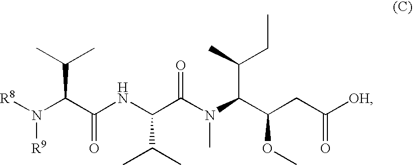

to produce the compound of formula (A).

### In this embodiment, R¹ and R³ in the above formulas are as defined herein before.

### R⁸ and R⁹ in the above formulas are each independently hydrogen or C₁₋₄ alkyl.

### R⁷ in the above formulas is phenylalkylamine, phenyldialkylamine, or phenylalkyloxy; wherein the alkyl group in phenylalkylamine, phenyldialkylamine, or phenylalkyloxy is a C₁₋₄ alkyl; and

the phenyl group in phenylalkylamine, phenyldialkylamine, or phenylalkyloxy may optionally be substituted with one, two or three substituents selected from the group consisting of halogen, alkoxycarbonyl, sulfamoyl, alkylcarbonyloxy, carbamoyloxy, cyano, mono- or di-alkylamino, alkyl, alkoxy, phenyl, phenoxy, trifluoromethyl, trifluoromethoxy, alkylthio, hydroxy, alkylcarbonylamino, 1,3-dioxolyl, 1,4-dioxolyl, amino and benzyl.

The reaction of the compound of formula (B) with the compound of formula (C) is known to the skilled artisan and well described inter alia in WO 03/008378.

Another embodiment of the present invention is the use of the above process in the manufacture of a compound of formula (A) as defined above.

Still another embodiment of the present invention is a process for making the compound of formula (A-1).

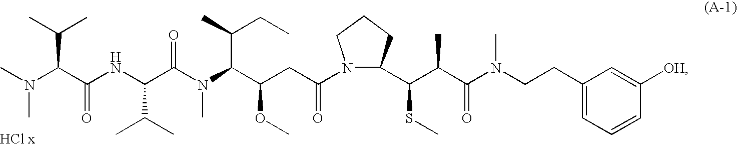

This process comprises the steps of:

(a) reacting the compound of formula (1a)

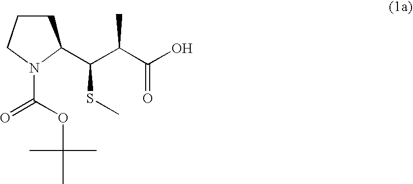

with 3-(2-methylamino-ethyl)-phenol;
(b) cleaving the tert-butoxycarbonate group contained in the resulting compound at the pyrrolidine N-atom, to produce the compound of formula (B-1),

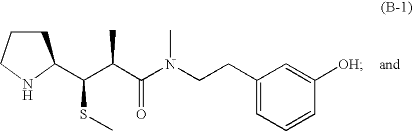

(c) reacting the compound of formula (B-1) with the compound of formula (C-1),

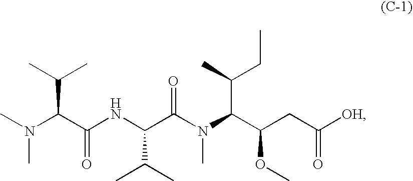

to produce the compound of formula (A-1).

Yet another embodiment of the present invention is the use of the above process in the manufacture of the compound of formula (A-1) as defined above.

The following examples are provided to aid the understanding of the present invention. It is understood that modifications can be made without departing from the spirit of the invention.

### EXAMPLES

If not explicitly otherwise stated, the following abbreviations are used:

min minute(s)
h hour(s)
d day(s)
eq. equivalents
rt room temperature

### NMR nuclear magnetic resonance

### GC gas chromatography

### TLC thin layer chromatography

### HPLC high performance liquid chromatography

dr diastereosiomer ratio
er enantiomer ratio
ee enantiomeric excess
mp melting point
sat. saturated

### TPPO triphenylphosphine oxide

aqu. aqueous

### TBME tert-butyl methyl ether

### Boc tert-butoxycarbonyl

### Ph phenyl

### Et ethyl

### Example 1

### Production of (2S)-2-(2-Ethoxycarbonyl-propenyl)-pyrrolidine-1-carboxylic acid tert-butyl ester (Boc-Dap-en-OEt, 2)

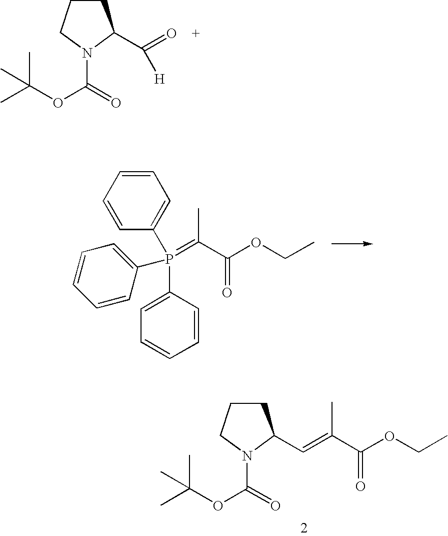

This example describes one method for producing Boc-Dap-en-OEt (2).

90.3 g (249 mmol) of the Wittig Ylide, ethyl-2-(triphenylphosphoranylidene)propionate, was suspended under argon and with stirring in 350 ml tert-butyl methyl ether, and 35.53 g Boc-L-prolinal (178 mmol) were added. The yellowish suspension was heated to reflux temperature. A yellowish solution formed first and after ca. 20 min of reflux the precipitation of triphenylphosphine oxide as a white solid started. After 2.5 h of reflux, the solvent was distilled off using a Dean Stark trap until the volume of the reaction mixture was reduced to about half of its original volume. Then 350 ml heptane were added while keeping the reaction mixture under reflux. The suspension was allowed to attain room temperature then further cooled and kept at 0-5° C. while stirring. The triphenylphosphine oxide precipitate was removed by filtration. The clear yellowish filtrate was evaporated (42° C./350 mbar) and the residue dried (rt/0.1 mbar) to produce 49.09 g of a pale yellow oil. The material by HPLC contained 81.1% (E)-2 and 7.2% (Z)-2 (E/Z=92:8). Filtration over silica gel with heptane/ethyl acetate 4:1 as the eluent followed by evaporation and drying in vacuo produced 46.32 g (92%) of the crude product as a clear white liquid.

¹H-NMR: (300 MHz, CDCl₃): 6.61 (d, br, J=8, olefinic H of E-isomer); 5.85 (br, olefinic H of Z-isomer); 5.05-4.3 (m, br, 1H); 4.19 (q, br, J=7, O—CH₂—CH₃); 3.6-3.35 (m, 2H); 2.35-2.05 (m, 1H); 2.0-1.75 (m, 5H); 1.67 (m, 1H); 1.46, s, br, 9H, t-Bu); 1.29 (t, J=7, O—CH₂—CH₃).

### Example 2

### Production of (2S)-2-(2-Ethoxycarbonyl-propenyl)-pyrrolidine-1-carboxylic acid tert-butyl ester (Boc-Dap-en-OEt, 2)

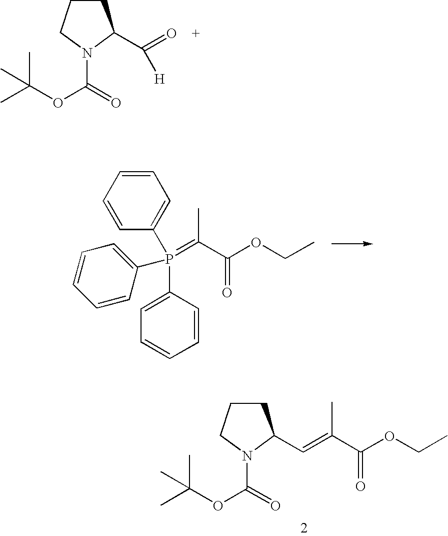

This example describes another method for producing Boc-Dap-en-OEt (2).

50.74 g (140 mmol) of the Wittig Ylide, ethyl-2-(triphenylphosphoranylidene)propionate, were suspended under argon and with stirring in 180 ml toluene and a solution of 19.92 g Boc-L-prolinal in 20 ml toluene was added. The light yellow suspension was stirred at 90° C. for 1 h to form first an almost clear solution then a white-yellow suspension. GC indicated almost complete consumption of Boc-L-prolinal. After cooling to room temperature, 200 ml heptane were added, which resulted in a milky emulsion, followed by addition of 100 ml methanol/water 7:3. The resulting two-phase system was transferred to a separatory funnel, the brownish aqueous phase was removed and the toluene/heptane phase washed successively with 100 ml methanol/water 7:3, 100 ml 10% aqu. citric acid solution and an additional 100 ml methanol/water mixture (7:3). The combined aqueous methanolic phases were back-extracted with 100 ml heptane. The combined toluene and heptane phases were washed with 100 ml 38% aqu. sodium bisulfite solution, 100 ml deionized water and 100 ml brine, dried over sodium sulfate, filtered and evaporated to yield 36.2 g of oil containing some solid triphenylphosphine oxide. The mixture was taken up in 30 ml heptane. The suspension was stirred for 5 min., then filtered over a bed of SPEEDEX® filter aid and the solid washed with 2×10 ml heptane. The combined filtrate and wash solutions were evaporated and the residue was dried in vacuo (0.1 mbar/rt/3 h) to provide as the crude product 26.05 g (91.8% by weight) of the title compound as a yellowish oily liquid. GC of the material revealed 6.7% (Z)-2 and 76.5% (E)-2 (E/Z=92:8).

¹H-NMR: (300 MHz, CDCl₃): 6.61 (d, br, J=8, olefinic H of E-isomer); 5.85 (br, olefinic H of Z-isomer); 5.05-4.3 (m, br, 1H); 4.19 (q, br, J=7, O—CH₂—CH₃); 3.6-3.35 (m, 2H); 2.35-2.05 (m, 1H); 2.0-1.75 (m, 5H); 1.67 (m, 1H); 1.46, s, br, 9H, t-Bu); 1.29 (t, J=7, O—CH₂—CH₃).

### Example 3

### Production of (2S)-2-(2-Ethoxycarbonyl-1-methylsulfanyl-propyl)-pyrrolidine-1-carboxylic acid tert-butyl ester (Boc-Dap-OEt, 4)

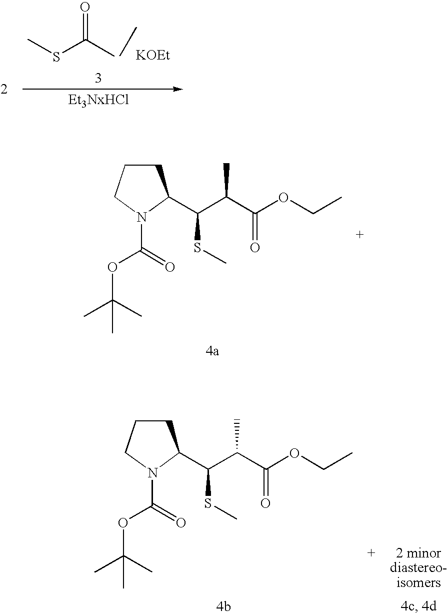

44.83 g of S-Methyl thioacetate (492 mmol) were dissolved under argon with stirring in 468 ml dry tetrahydrofuran. To the clear colorless solution 41.4 g potassium ethoxide (492 mmol) were added at once through a glass funnel. The temperature of the orange-brownish suspension rose to 38° C. The suspension was stirred at rt for 2 h. The transesterification reaction was monitored by GC. Then 34.0 g triethylamine hydrochloride (246 mmol) were added at once followed by dropwise addition of a solution of 46.32 g Boc-Dap-en-OEt (2) as obtained from Example 1 in 150 ml dry tetrahydrofuran. The orange-brownish suspension was stirred at room temperature for 20 h. The reaction was monitored by TLC. After 22 h, 150 ml ethyl acetate and 320 ml 5M ammonium chloride solution were added to the reaction mixture. The two phase system was stirred at room temperature for 5 min, then separated with a seperatory funnel. The organic phase was dried over sodium sulfate, filtered and evaporated (42° C./300 mbar) to yield as the crude product 54.6 g of the title compound as orange-brown oil. Filtration over the five-fold amount of silica gel, i.e. 273 g SiO₂ with heptane/ethyl acetate (4:1) as the eluent followed by evaporation and drying in vacuo afforded the title product in 3 fractions. The 3 fractions were combined to produce 52.65 g (89.2% based on Boc-L-prolinal) of Boc-Dap-OEt (4) as a pale yellow oil. GC revealed a composition that was 80.44% diastereoisomer 4a, 2.44% diastereoisomer 4c, diastereoisomer 8.90% 4b, and 3.60% diastereoisomer 4d. The diastereomeric ratio was determined to be 84.4:9.3:2.6:3.8 (4a:4b:4c:4d).

¹H-NMR: (300 MHz, CDCl₃): 4.15 (m, O—CH₂—CH₃); 4.05-3.1 (m, br, 4H); 2.56 (m, 1H); 2.11 (s, SCH₃); 2.0-1.8 (m, br, 3H); 1.8-1.65 (m, br, 1H); 1.49, s, br, 9H, t-Bu); 1.33 (d, J=7, —CH—CH₃); 1.28 (t, J=7, O—CH₂—CH₃).

### Example 4

### Production of (S)-2-(1R,2S)-2-Carboxy-1-methylsulfanyl-propyl)-pyrrolidine-1-carboxylic acid tert-butyl ester (Boc-Dap-OH, 1a)

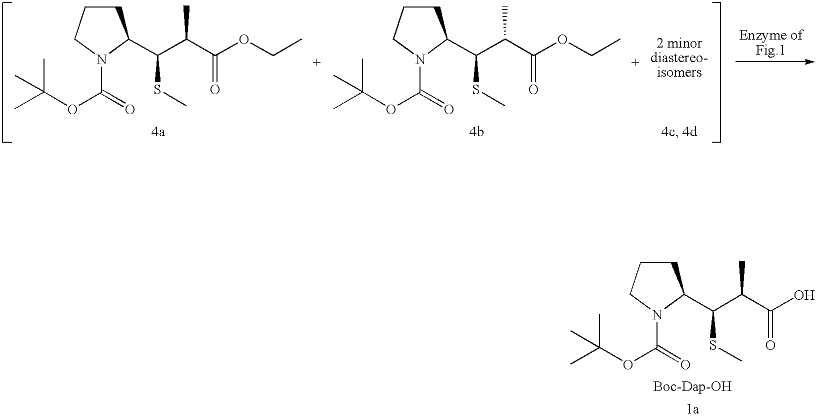

An emulsion of 28.90 g Boc-Dap-OEt (4), as obtained from Example 3, in 1450 ml 5 mM potassium phosphate buffer (pH 7.5) with 0.1 M sodium chloride (2% substrate concentration) was heated to 30° C. under stirring. Then 656 mg esterase having the amino acid sequence of SEQ ID NO: 1 were added and the stirred emulsion was kept at pH 7.5 and 30° C. by the controlled addition of 1.0 M sodium hydroxide solution. After consumption of 74.81 ml of said sodium hydroxide solution (74.8 mmol≅0.86 eq.) the reaction terminated and the reaction mixture was extracted with 700 ml tert-butyl methyl ether. The organic phase washed with 500 ml saturated sodium hydrogencarbonate solution. The combined aqueous phases were adjusted to pH 1.5 with ˜40 g concentrated sulfuric acid, and the white suspension formed was extracted with 2×1400 ml ethyl acetate. The combined ethyl acetate phases were dried with ˜100 g sodium sulfate, filtered and evaporated. Drying over night in vacuo gave as the crude product 21.71 g Boc-Dap-OH (1a) as light yellow viscous oil. The diastereomeric ratio was determined by GC to be 99.7:0.14:0.0:0.2 (1a:1b:1c:1d), with 1b, 1b, 1c, and 1d, as discussed herein, being the products of the above enzymatic reaction when the substrate is the diastereoisomer 4a, 4b, 4c, and 4d, respectively.

¹H-NMR: (300 MHz, CDCl₃): 4.09 and 4.00 (2 m, NCH of 2 rotamers); 3.56 and 3.45 (2 m, CHS of 2 rotamers); 3.20 (br. m, NCH₂); 2.11 (s, SCH₃), 1.94 and 1.77 (2 m, CCH₂CH₂C); 1.47 and 1.45 (2 s, tBu of 2 rotamers), 1.39 (br. d, J=6.2, CH₃)).

Crystallization 21.3 g of the crude product were dissolved in 104 ml n-hexane under stirring at ˜42° C. After 4 h at room temperature the addition of 1 mg seeding crystals started the crystallization. After 3d at 4° C. the crystals were filtered off, washed with 10 ml pre-cooled n-hexane (−20° C.) and dried over night in vacuo to give as 1^{st} crop material 14.85 g of the diastereoisomer 1a (51% based on Boc-L-prolinal) as white crystals. The diastereomeric ratio of the product was determined by GC to be 100:0:0:0 (1a:1b:1c:1d).

¹H-NMR: not distinguishable from the NMR above.

The residue from the mother liquor (4.7 g yellow oil) was dissolved in 22 ml n-hexane under stirring at ca. 42° C. After 4 h at room temperature the addition of 1 mg seeding crystals started the crystallization. After 3 d at 4° C. the crystals were filtered off, washed with 5 mL pre-cooled n-hexane (−20° C.) and dried over night in vacuo to give as 2^{nd} crop material 1.5 g of the diastereoisomer 1a as white crystals. The diastereomeric ratio was determined by GC to be 99.85:0:0:0.15 (1a:1b:1c:1d).

¹H-NMR: not distinguishable from the NMR above Combined yield: 16.35 g (63% by weight; 56% based on Boc-L-prolinal over 3 steps) of the title compound (1a).

### Example 5

### Production of (S)-2-(1R,2S)-2-Carboxy-1-methylsulfanyl-propyl)-pyrrolidine-1-carboxylic acid tert-butyl ester (Boc-Dap-OH, 1a)

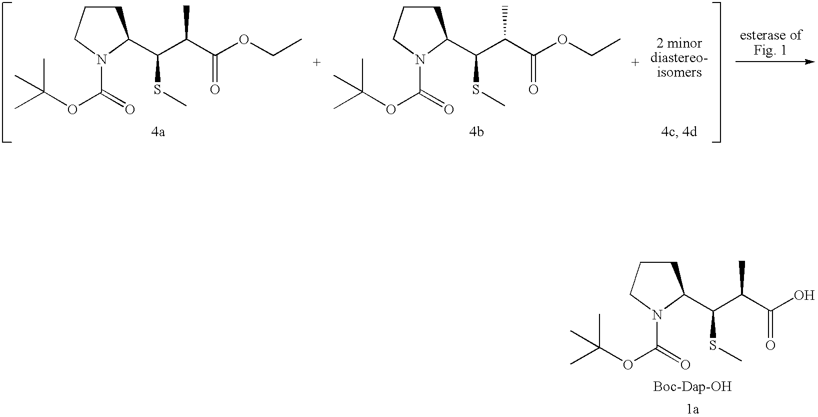

An emulsion of 12.80 g Boc-Dap-OEt (4), synthesized analog to Example 3 (diastereomeric ratio of 4a/4b/4c/4d=85.1:8.4:2.7:3.8), and 42.6 g PEG6000 (Fluka) in 370 ml 5 mM potassium phosphate buffer (pH 7.5) with 0.1 M sodium chloride (3% substrate concentration) was heated to 30° C. under stirring. Then 129 mg esterase of the sequence of SEQ ID NO: 1 were added and the stirred emulsion was kept at pH 7.5 and 30° C. by the controlled addition of 1.0 M sodium hydroxide solution. After 6d-termination of the reaction, HPLC control—the reaction mixture was extracted with 250 ml tert-butyl methyl ether. The organic phase washed with 250 ml saturated sodium hydrogencarbonate solution. The combined aqueous phases were adjusted to pH 1.5 with concentrated sulfuric acid, and the white suspension formed was extracted with 2×500 ml ethyl acetate. The combined ethyl acetate phases were dried with ˜60 g sodium sulfate, filtered and evaporated. Drying over night in vacuo gave as the crude product 8.89 g Boc-Dap-OH (1a) as colorless viscous oil. Crystallization occurred spontaneously and was allowed to proceed for further 24 h at room temperature and −20° C. After filtration, washing with ˜10 ml cold pentane (Fluka) and drying 7.4 g Boc-Dap-OH (1a) as colorless crystals was obtained. The diastereomeric ratio was determined by GC to be 99.64:0.06:0.04:0.27 (1a:1b:1c:1d).

¹H-NMR: not distinguishable from the NMR of example 4

1. A process for making a compound of formula (I) 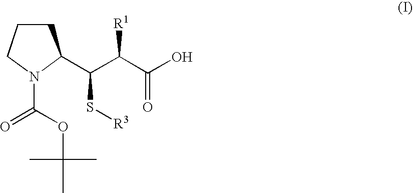
comprising the steps of:
(A) reacting a compound of formula (II) 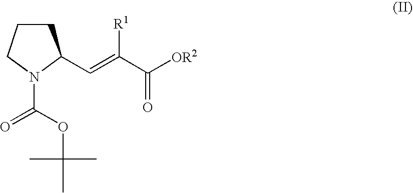
with a compound of formula (III)
KS—R³ (III),
in the presence of triethylammonium chloride in a suitable solvent to obtain a diastereomeric mixture of formula (IV) 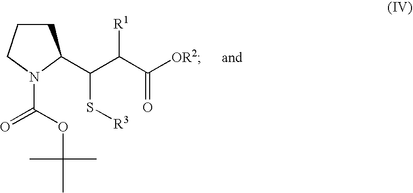
(B) cleaving, using a hydrolase, R² from the —COOR² ester group of the diastereoisomer of the above diastereomeric mixture that has the formula of formula (IVa) 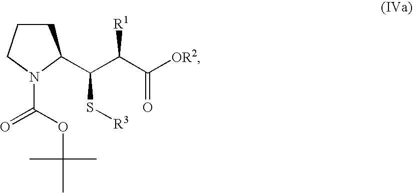
to produce a compound of formula (I);
R¹ in the above formulas is methyl or ethyl which can be substituted, once or several times, by fluorine; or unsubstituted propyl;
R² in the above formulas is alkyl;
R³ in the above formulas is methyl or ethyl.

2. A process according to claim 1 wherein said compound of formula (III) is generated in situ by reacting a compound of formula (III-A), 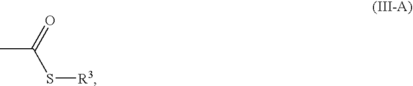
in which R³ is methyl or ethyl, in the presence of a potassium base.

3. A process according to claim 1, wherein said hydrolase is an esterase.

4. A process according to claim 3, wherein said esterase is a stereoselective esterase.

5. A process according to claim 4, wherein said esterase has the amino acid sequence of SEQ ID NO: 1 or is a variant thereof.

6. A process according to claim 4, wherein said esterase is a protein that is encoded by the nucleic acid sequence SEQ ID NO: 2 or is a variant of such a protein.

7. A process according to claim 1, wherein
R¹ is methyl;
R² is methyl, ethyl, propyl, or butyl;
R³ is methyl; and
said hydrolase is an esterase having the amino acid sequence of SEQ ID NO: 1.

8. A process according to claim 2, wherein
R¹ is methyl;
R² is methyl, ethyl, propyl, or butyl;
R³ is methyl; and
said hydrolase is an esterase having the amino acid sequence of SEQ ID NO: 1.

9. A process according to claim 1, wherein R¹ and R³ are both methyl.

10. A process according to claim 9, wherein R² is ethyl.

11. A process for making a compound of formula (A), 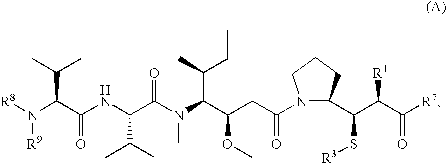
comprising:
(A) reacting a compound of formula (II) 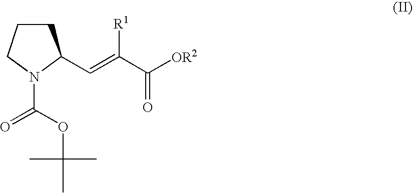
with a compound of formula (III)
KS—R³ (III),
in the presence of triethylammonium chloride in a suitable solvent to obtain a diastereomeric mixture of formula (IV) 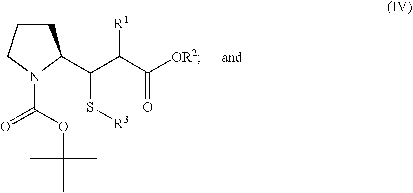
(B) cleaving, using a hydrolase, R² from the —COOR² ester group of the diastereoisomer of the above diastereomeric mixture that has the formula of formula (IVa) 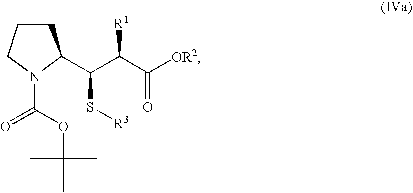
to produce a compound of formula (I), 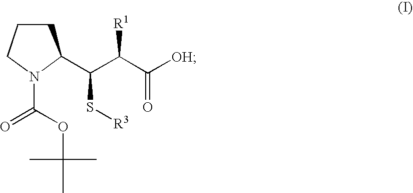
(C) reacting said compound of formula (I) with a compound of the formula R⁷H;
(D) cleaving the tert-butoxycarbonyl group contained in the resulting compound at the pyrrolidine N-atom, to produce a compound of formula (B), 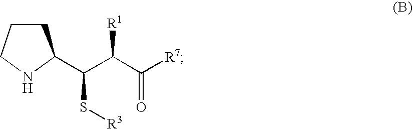
and (E) reacting said compound of formula (B) with a compound of formula (C), 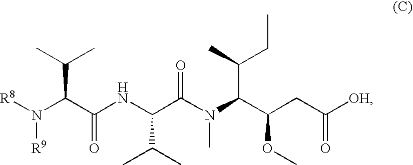
to produce a compound of formula (A);
R¹ in the above formulas is methyl or ethyl which can be once or several times substituted by fluorine; or unsubstituted propyl;
R² in the above formulas is alkyl;
R³ in the above formulas is methyl or ethyl;
R⁸ and R⁹ in the above formulas are each independently hydrogen or C₁₋₄alkyl; and
R⁷ in the above formulas is phenylalkylamine, phenyldialkylamine, or phenylalkyloxy;
wherein the alkyl group in phenylalkylamine, phenyldialkylamine, or phenylalkyloxy is a C₁₋₄ alkyl; and
the phenyl group in phenylalkylamine, phenyldialkylamine, or phenylalkyloxy may optionally be substituted with one, two or three substituents selected from the group consisting of halogen, alkoxycarbonyl, sulfamoyl, alkylcarbonyloxy, carbamoyloxy, cyano, mono- or di-alkylamino, alkyl, alkoxy, phenyl, phenoxy, trifluoromethyl, trifluoromethoxy, alkylthio, hydroxy, alkylcarbonylamino, 1,3-dioxolyl, 1,4-dioxolyl, amino and benzyl.

12. The process according to claim 11 wherein: said compound of formula (A) is a compound of formula (A-1), 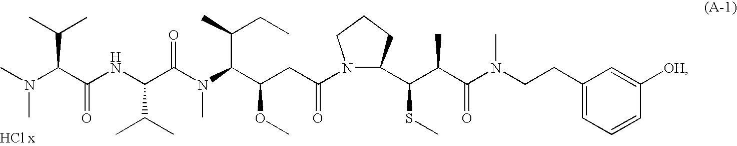
R¹ and R³ are both methyl;
R⁷ is 3-(2-methylamine-ethyl)-phenol;
said compound of formula (B) is the compound of formula (B-1), 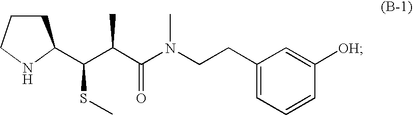
and said compound of formula (C) is the compound of formula (C-1) [chemical structure]


SEQUENCE LISTING (2 sequences)

SEQ ID NO: 1 - protein, 371 aa, Artificial Sequence
  Description of Artificial Sequence Synthetic amino acid sequence
MTTSTQHISELPLLPGRLGDPDRVLKTDPRADPRLVAACAPFALDVAPPPAPVNAHSPLQDKLDYSAANEAGMETVFAALFADLPPMTNVERRTEVIKGVDGNDIKLYIHTPQHVSGPLPCVYHIHGGGMVILTAAGPTYVRWRDELAALGMVVVGVEFRNGAGKLGNHPFPAGLNDCMSGLQWTFDHKTTLGISKIIVSGESGGGNLSLAVCLKAKKDKRLEQIHGVYALCPYIYGAWAQKSKDLPSLYENDGYLINCSLMEVLASVYDPEGKNATNPLCWPYWATREDLQGLPPHVISVNELDPLRDEGLKYYQRLLAAGVRVYSRTVNGTCHAGDVLFRKALPDVYAATLRDIKGFADSLGSHHHHHH

SEQ ID NO: 2 - DNA, 1116 bp, Artificial Sequence
  Description of Artificial Sequence Synthetic nucleotide sequence
atgaccacatctacacaacacatcagcgagctacctctcttaccaggccgtctcggcgaccccgatcgcgtcttgaagactgatccccgtgccgatcctcggttggtcgccgcctgcgccccctttgctttagacgttgcgcccccacccgcaccggtgaacgcgcactcgcccttgcaggacaagctcgactacagtgcggccaacgaggcgggcatggaaaccgtgtttgccgccctgttcgccgacctcccgccgatgacgaacgtggagcggcggaccgaggtcatcaagggcgtggacggtaacgacatcaagctgtatatccatacgccccagcacgtttctggcccgctgccctgcgtgtatcatatacacggcggcggcatggtcattctgacggccgctggccctacctatgtacgctggcgggacgagcttgccgccctgggcatggtcgtggtgggtgtggaattccgtaatggcgccggcaagcttggcaaccatccctttccagccgggctcaacgactgcatgagtggcttacagtggacgtttgaccacaaaaccaccctggggatctcgaagataatcgtgtccggtgagtctggtggaggcaatctctccctggccgtgtgcctcaaggccaagaaggacaaacgccttgagcagattcatggtgtctacgccttgtgtccgtacatttatggcgcatgggcgcagaagagcaaagatctcccttccctatacgaaaacgacggctacttgatcaactgtagcctgatggaggtgctggcaagcgtctatgaccctgaaggcaaaaacgccaccaatccgctgtgctggccatactgggccacacgtgaggacctgcaaggtctgcctccgcatgtgatctcggtcaacgagttagaccccctgcgagacgaggggctgaaatactatcagaggctcctggcggctggggtgcgtgtatacagccggaccgtcaacggcacgtgccatgcgggcgacgtcctgttccgcaaagcgctccctgacgtgtacgcagccaccctccgcgatatcaaggggttcgcagactcgctgggatctcatcaccatcaccatcactaa